(19) United States
(12) Patent Application Publication
(10) Pub. No.: US 2026/0216240 A1
(43) Pub. Date: Jul. 30, 2026

(54) ANTI-INFLAMMATORY COMPOSITION COMPRISING COLLOIDAL SILVER AND PLANT EXTRACTS

(71) Applicant: BOBS SILVER, Paris (FR)

(72) Inventor: Sadek BAITI, Fougeres (FR)

(21) Appl. No.: 19/151,896

(22) PCT Filed: Jan. 31, 2024
(86) PCT No.: PCT/EP2024/052370

§ 371 Date: Jul. 29, 2025

(30) Foreign Application Priority Data
Jan. 31, 2023 (FR) FR2300874

(51) Int. Cl. A61K 33/38; A61K 8/19; A61K 8/9789; A61K 35/644; A61K 36/185; A61K 36/28; A61K 36/282; A61K 36/38; A61K 36/8962; A61P 29/00; A61Q 19/00

(52) U.S. Cl. CPC A61K 33/38; A61K 8/19; A61K 8/9789; A61K 35/644; A61K 36/185; A61K 36/28; A61K 36/282; A61K 36/38; A61K 36/8962; A61P 29/00; A61Q 19/00

(57) ABSTRACT

The invention relates to a composition for use as medication in inflammation prophylaxis and/or treatment comprising at least silver in colloidal form, Sambucus Nigra extract, Primulae flos cum calycibus extract and/or Hypericum perforatum extract, and Pelargonium sidoides extract. According to an embodiment, said composition is used for prophylaxis and/or treatment of Staphylococcus aureus-inducedl inflammation.

The invention relates to a composition for use as medication for inflammation prophylaxis and treatment comprising at least silver in colloidal form, Sambucus Nigra extract, Hypericum perforatum extract and Pelargonium sidoides extract. According to an embodiment, said composition is used for prophylaxis and treatment of Staphylococcus aureus-induced inflammation.

The invention relates to inflammation prophylaxis and treatments, in particular induced by Staphylococcus aureus.

### PRIOR ART

Knowledge of the healing properties of silver dates back to the Ancient Greek civilization:
families using silver cutlery were known to be less prone to infection. Silver has a number of antimicrobial properties as the presence of one part per 100 million elemental silver in a solution can achieve an effective antimicrobial effect. It is also a well-known fact that silver ions and free radicals are an active antimicrobial agent.

To make silver water-soluble, a colloidal ionic form (extremely fine silver particles suspended in water) must be used, produced for the first time in 1920 through hydrolysis wherein two pure silver electrodes, submerged in demineralized or distilled water, were used, and to which a voltage difference was applied.

Prior to the advent of antibiotics in 1938, colloidal silver obtained using this process was prescribed for a wide variety of diseases and infections: indeed, it was proven that it was efficient against more than 650 different pathogens.

Colloidal silver boosts the immune system, acting as a catalyst by deactivating the specific enzymes required for the metabolism of a wide variety of bacteria, fungi and viruses.

Its success is due to the fact that it is a safe antiseptic. Silver, when used in colloidal form, is very slowly absorbed by tissue to prevent any toxic effects. Therefore, it can be taken orally and can be used in preparations taken daily by both adults and children, without any risk of causing any intolerance, even over a long period of time.

Colloidal silver is primarily used as an antiseptic in dermatology in the treatment of certain lesions. It is also known for its application as water purification agent and a preservative in the cosmetics industry.

However, the efficacy of colloidal silver does not cover all fungi, viruses and bacteria. Staphylococcus aureus is a major pathogen that causes a variety of bacterial skin infections (Liu et al., 2018; Tatiya-Aphiradee et al., 2019). This bacteria is the most prominent staphylococcus strain in human and veterinary medicine. Along with Escherichia coli, this bacterium is well known for being a primary cause of healthcare-associated infections. In France, S. aureus is also one of the main bacteria responsible for food intoxications.

Inflammation is described as a reaction resulting from two conditions: a pathological condition or a defense response of the host against pathogens (Medzhitov, 2008; Gu-nalan et al., 2014; Greten, & Grivennikov, 2019; Agarwal, & Shanmugam, 2020). It is a well-known fact that long-term inflammation can lead to the onset of rheumatoid arthritis, inflammatory bowel diseases, asthma and pancreatitis (Mueller et al., 2010) and increases the risk of cancer (Agarwal, & Shan-mugam, 2020). An increase in cytokine levels, cytokine receptors, adhesion molecules, immunoregulatory factors and a number of other mediators is the key mechanism of inflammation (Gunalan et al., 2014). As chronic inflammation can lead to permanent effects, a number of studies have focused on developing anti-inflammatory products. Unfortunately, a number of commercially-available drugs are associated with side effects. It is a well-known fact that the IL-1 family is involved in acute and chronic inflammation (Kolb et al., 2001) and thus plays a significant role in controlling inflammation processes (Sims et Smith, 2010). The IL-1 gene family is characterized by three constituents-IL-1α, IL-1β and the IL-1 receptor antagonist (IL-1RA) (Dinarello, 1997). IL-1β is released following a proteolytic cleavage and is very likely to be involved in the inflammatory process (Kolb et al., 2001) as its over expression and the subsequent inflammatory cascade can lead to the development of an auto-immune condition (Pa-thak et al., 2011). Considering the physiopathological relevance of IL-1β, a number of studies focused on developing drugs and products that target this interleukin (Peiró et al., 2017) were conducted. To date, a number of drugs have been approved. These include anakinra, rilonacept and canakinumab (Peiró et al., 2017).

Furthermore, the over expression and upregulation of IL-8 are reportedly linked to a number of cancers, including breast cancer (Singh et al., 2013). However, studies have shown a significant increase in post-menopausal women suffering from osteoporosis and bone loss (Al-Daghri et al., 2014). Therefore, a decrease in this marker can significantly improve these health issues.

An efficient alternative to currently existing anti-inflammatory compositions that mitigates associated side effects is yet to be found. In particular, the need for prophylactic inflammation treatment, in particular related to Staphylococcus aureus-induced infections, still remains.

Unexpectedly, the applicant highlighted in an unexpected manner that the combination of colloidal silver with several plant extracts produced an efficient composition for inflammation prophylaxis and/or treatment, in particular Staphylococcus aureus-induced inflammation. In fact, the applicant identified that the composition according to the invention could reduce the production of inflammatory cytokines.

### DESCRIPTION

Therefore, according to a first aspect, the invention relates to a composition for use as medication for inflammation prophylaxis and/or treatment comprising:
Silver in colloidal form;
Sambucus Nigra extract;
Hypericum perforatum extract;
Pelargonium sidoides extract.

According to the invention, ionic colloidal silver means extremely fine silver particles suspended in water.

Preferably, said silver in colloidal form has a particle size between 0.1 and 30 nanometers.

Preferably, said composition for use according to the invention comprises:
Between 20 mg and 250 mg of silver in colloidal form per liter of composition.

By convention, it is generally considered that 1 mg of silver equates to 1 ppm in a 1 Liter solution, i.e. 20 mg to 250 mg per liter of solution.

Sambucus Nigra, also known as Elderberry, is a shrub of the Caprifoliaceae family. Dry, aqueous and/or alcoholic plant extracts can be derived from its flowers or its fruit.

Preferably, said composition for use according to the invention comprises:
Between 0.3 and 5% by weight of the Sambucus Nigra Extract composition.

Hypericum perforatum, also known as Saint John's Wort, is a perennial herbaceous plant of the Clusiceae family. Said extracts are derived from the plant or flower tops that can be crushed, optionally in water and/or alcohol.

Preferably, said composition for use according to the invention comprises:
Between 0.5 and 11% by weight of the Hypericum perforatum extract composition;

Pelargonium sidoides, also known as Umckaloabo, is a plant native to South Africa. Extracts of this plant can be dry, aqueous and/or alcoholic extracts, derived from the roots or other plant parts.

Preferably, said composition for use according to the invention comprises:
Between 1 and 6% by weight of the Pelargonium sidoides Extract composition;

According to the invention, “treatment” means the administration of the composition according to the invention to a patient with a health condition, in order to help them heal, relieve their symptoms, inflammation in this particular case under this invention, preferably in relation with S. aureus-induced infection.

According to the invention, “prophylaxis” means the execution of the composition according to the invention for an individual or patient to prevent the onset or spreading of a health condition, inflammation in this particular case under this invention, preferably in relation with S. aureus-induced infection.

“Inflammation” is the body's defense response to an attack. Inflammation can be acute or chronic. The clinical signs of inflammation are pain (pulsing), redness, swelling (edema), feeling hot and functional impotence. Histologically, in acute forms, there is a sequence of local vascular changes (vasodilatation with slower blood circulation, increased permeability and edema), then a surge of circulating leukocytes, initially granulocytes, then monocytes with margination, adherence to endothelial cells, diapedesis, phagocytosis of foreign substances and cell and tissue debris and possibly pus formation. The development of the inflammatory response is variable. In acute inflammation, following the elimination of the causal agent, mononuclear phagocytes (monocytes, macrophages, histiocytes, etc.) clean the inflammatory site, and either “ad integrum restitution” without any aftereffects occurs, or the destroyed tissue is replaced by a scar fibrosis without functional restitution. In other cases, if the root cause is not eliminated or due to a failure in certain body defense mechanisms, inflammation becomes chronic, characterized by the formation of a generally mixed inflammatory infiltrate (granulocytes, histiocytes-macrophages, lymphocytes, and even giant cells and epithelioids) with neo-angiogenesis, and especially, tissue change processes (with combined destruction and reconstruction) with significant fibroblastic proliferation in particular.

A large number of cell types (mononuclear and polynuclear phagocytes, lymphocytes, platelets, as well as tissue cells, endothelial cells, etc.) and humoral mediators (complement, contact system, hemostasis protein), all therapeutic targets, are involved in the inflammatory response. Finally, most inflammatory responses induce a systemic response comprising increased plasma protein concentrations called of the acute phase (such as C-reactive protein, serum amyloid A protein, several α-1-globulins, certain complement components, fibrinogen, haptoglobin and a decrease in other protein concentrations such as albumin, transferrin, fibronectin, apoproteinA-1. Interleukins are also involved in the inflammatory response, including the IL-1 family (IL-1α, IL-1β and the IL-1 receptor antagonist (IL-1RA) (Dinarello, 1997)), which plays a significant role in controlling inflammation processes (Sims et Smith, 2010). IL-8 is also an interleukin known for being involved in inflammation.

The composition for use according to the invention reduces the production of inflammatory cytokines, IL-8, IP-10, IL-1β and IL-6 in particular.

According to an embodiment, said composition for use according to the invention comprises:
Between 20 mg and 250 mg of silver in colloidal form per liter of composition;
Between 0.3 and 5% by weight of the Sambucus Nigra extract composition;
Between 0.5 and 11% by weight of the Hypericum perforatum extract composition;
Between 1 and 6% by weight of the Pelargonium sidoides extract composition.

According to an embodiment, said composition for use according to the invention further comprises: 5
Silver in ionic form;

According to the invention, ionic silver means silver whose particles have been dissolved in water, as opposed to colloidal silver where said particles are suspended in water.

According to an embodiment, said composition for use according to this latter embodiment comprises:
Between 20 mg and 250 mg of total silver per liter of composition, distributed in
   i. Between 70 and 99.99% by total weight of silver, silver in
   ii. Between 0.01 and 30% by total weight of silver, silver in ionic form;

According to the invention, total silver means all the silver included in the composition according to the invention, irrespective of its form.

According to an embodiment, said composition for use comprises:
Between 20 mg and 250 mg of total silver per liter of composition;
   i. Between 70 and 99.99% by total weight of silver, silver in colloidal form;
   ii. Between 0.01 and 30% by total weight of silver, silver in ionic form;
Between 0.3 and 5% by weight of the Sambucus Nigra extract composition;
Between 0.5 and 11% by weight of the Hypericum perforatum extract composition;
Between 1 and 6% by weight of the Pelargonium sidoides extract composition.

According to an embodiment, said composition for use according to the invention further comprises:
Echinacea extract, and/or
Propolis and/or
Artemisia extract and/or
Allium sativum extract and/or
Primulae flos cum calycibus extract.

Primulae flos cum calycibus, also known as Cowslip, is a perennial herb of the Primulaceae family. Said extracts are derived from crushed flowers and/or calyx, optionally in water and/or alcohol. These extracts can also be dry extracts.

Preferably, said composition for use according to the invention comprises:
Between 1 and 9% by weight of the Primulae flos cum calycibus Extract composition.

Echinacea refers to a plant genus of the Asteraceae family. Said extracts are dry, aqueous and/or alcoholic extracts, derived from the root, the whole plant or aerial plant parts.

Preferably, said composition for use according to the invention comprises:
Between 0.01 and 8% by weight of the Echinacea extract composition.

Allium sativum, a plant more commonly known as “Garlic” is a perennial plant species. Extracts can be in dry, aqueous and/or alcoholic form, or in aged garlic extract form.

Preferably, said composition for use according to the invention comprises:
Between 0.01 and 10% by weight of the Allium sativum extract composition.

“Propolis” is a product derived from plant resins and exsudates, which can be secreted by plants or produced by bees from plant resin and wax. As propolis depends on the plant species from which it is produced, standardized propolis is produced, often defined by its color. Preferably, yellow, green or red propolis is used. Said propolis can be in dry, purified, aqueous and/or alcoholic extract form.

Preferably, said composition for use according to the invention comprises:
Between 0.01 and 7% by weight of the propolis composition.

Artemisia, also known as Mugwort, is a genus of plants comprising herbs and shrubs, featuring pinnate leaves. Preferably, the species used according to the invention is Artemisia annua. It is an aromatic branched herb. Extracts from this plant can be derived from the whole plant or its aerial parts, and may be dry, aqueous and/or alcoholic extracts.

Preferably, said composition for use according to the invention comprises:
Between 0.5 and 3% by weight of the Artemisia extract composition;

Preferably, said composition for use according to the invention comprises:
Between 0.01 and 8% by weight of the Echinacea extract composition, and/or
Between 0.01 and 7% by weight of the Propolis composition, and/or
Between 0.5 and 3% by weight of the Artemisia extract composition, and/or
Between 0.01 and 10% by weight of the Allium sativum extract composition, and/or
Between 1 and 9% by weight of the Primulae flos cum calycibus extract composition.

Preferably according to the invention, said composition for use according to the invention comprises at least one other dermatologically and/or pharmaceutically acceptable agent.

According to an embodiment, said composition for use according to the invention is intended to be used as medication in the prophylaxis and/or treatment of Staphylococcus aureus-induced inflammation.

Staphylococcus aureus is a gram-positive bacterium characterized by its rapidly increasing resistance to antibiotics (Tatiya-Aphiradee et al., 2019).

Preferably, said composition for use according to the invention can be administered via the oral, intravenous, subcutaneous, nasal, ocular, otic, rectal, vaginal, respiratory route or can be applied to the skin.

Even more preferably, the oral route can be in the form of a tablet, syrup, drinkable solution or suspension, or capsule, preferably a tablet.

Even more preferably, the intravenous route can be a solution.

Even more preferably, the subcutaneous route can be a solution.

Preferably, administration is done by topical application.

Even more preferably, the topical application route can be an ointment, cream, patch, liquid solution or gel.

Even more preferably, the topical application route is a cream or a liquid solution.

Preferably, said composition for use according to the invention is administered through the respiratory or nasal route, as nose drops, nasal spray, nasal powder, spray, for example compressed gas nose sprays, or a nebulizer.

Advantageously, when the composition is suitable for nasal or respiratory administration, it can advantageously be used for broncho-pulmonary or sinus purposes.

Preferably, said composition for use according to the invention is administered by a spray through the nose.

The composition according to this invention can be administered to a patient on a daily basis, once daily, twice daily, three times daily or even more times a day.

Preferably, the composition of this invention is administered to a patient once daily.

The composition according to the invention can also comprise a physiologically acceptable medium, i.e. compatible with the skin or the other methods of administration considered.

According to an embodiment of the invention, said composition comprises, by weight of composition:
Purified Water for pharmaceutical use: qs
A total of 20 mg of silver, up to 20% ionic silver and 80% colloidal silver, having a mean particle size of 0.8nm;
0.9% Sambucus Nigra extract;
5% Hypericum perforatum extract;
1.5% Pelargonium sidoides dry extract;
3% Echinacea dry extract;
4% Propolis purified extract.

According to an embodiment of the invention, said composition comprises, by weight of composition:
Purified Water for pharmaceutical use: qs
A total of 20 mg of silver, up to 20% ionic silver and 80% colloidal silver, having a mean particle size of 0.8nm;
0.9% Sambucus Nigra extract;
5% Primulae flos cum calycibus dry extract;
5% Hypericum perforatum extract;
1.5% Pelargonium sidoides dry extract;

3% Echinacea dry extract;
4% Propolis purified extract.

According to an embodiment of the invention, said composition comprises, by weight of composition:
A total of 20 mg of silver, up to 20% ionic silver and 80% colloidal silver, having a mean particle size of 0.8 nm.
0.9% Sambucus Nigra extract.
5% Hypericum perforatum dry extract.
1.5% Pelargonium sidoides dry extract.
3% Echinacea dry extract.
4% Propolis purified extract.

According to a second aspect, the invention relates to a cosmetic or dermatological composition comprising:
Silver in colloidal form;
Sambucus Nigra extract;
Hypericum perforatum extract;
Pelargonium sidoides extract.

Preferably, said cosmetic or dermatological composition according to the invention comprises:
Between 20 mg and 250 mg of silver in colloidal form per liter of composition.

Preferably, said cosmetic or dermatological composition according to the invention comprises:
Between 0.3 and 5% by weight of the Sambucus Nigra Extract composition.

Preferably, said cosmetic or dermatological composition according to the invention comprises:
Between 0.5 and 11% by weight of Hypericum perforatum extract.

Preferably, said cosmetic or dermatological composition according to the invention comprises:
Between 1 and 6% by weight of the Pelargonium sidoides Extract composition.

According to an embodiment, said cosmetic or dermatological composition according to the invention comprises:
Between 20 mg and 250 mg of silver in colloidal form per liter of composition;
Between 0.3 and 5% by weight of the Sambucus Nigra Extract composition;
Between 0.5 and 11% by weight of the Hypericum perforatum extract composition;
Between 1 and 6% by weight of the Pelargonium Sidoides Extract composition.

According to an embodiment, said cosmetic or dermatological composition according to the invention further comprises:
Silver in Ionic Form;

According to an embodiment, said cosmetic or dermatological composition according to this latter embodiment comprises:
Between 20 mg and 250 mg of total silver per liter of composition, distributed in
   i. Between 70 and 99.99% by total weight of silver, silver in colloidal form;
   ii. Between 0.01 and 30% by total weight of silver, silver in ionic form;

According to an embodiment, said cosmetic or dermatological composition comprises:
Between 20 mg and 250 mg of total silver per liter of composition, distributed in
   i. Between 70 and 99.99% by total weight of silver, silver in colloidal form;
   ii. Between 0.01 and 30% by total weight of silver, silver in ionic form;
Between 0.3 and 5% by weight of the Sambucus Nigra Extract composition;
Between 0.5 and 11% by weight of the Hypericum perforatum extract composition;
Between 1 and 6% by weight of the Pelargonium Sidoides Extract composition.

According to an embodiment, said cosmetic or dermatological composition according to the invention further comprises:
Echinacea extract, and/or
Propolis and/or
Artemisia extract and/or
Allium sativum extract and/or
Primulae flos cum calycibus extract.

Preferably, said cosmetic or dermatological composition according to the invention comprises:
Between 1 and 9% by weight of the Primulae flos cum calycibus Extract composition.

Preferably, said cosmetic or dermatological composition according to the invention comprises:
Between 0.01 and 8% by weight of the Echinacea extract composition.

Preferably, said cosmetic or dermatological composition according to the invention comprises:
Between 0.01 and 10% by weight of the Allium sativum extract composition.

Preferably, said cosmetic or dermatological composition according to the invention comprises:
Between 0.01 and 7% by weight of the propolis composition.

Preferably, said cosmetic or dermatological composition according to the invention comprises:
Between 0.5 and 3% by weight of the Artemisia extract composition.

Preferably, said cosmetic or dermatological composition according to the invention comprises:
Between 0.01 and 8% by weight of the Echinacea extract composition, and/or
Between 0.01 and 7% by weight of the Propolis composition, and/or
Between 0.5 and 3% by weight of the Artemisia extract composition, and/or
Between 0.01 and 10% by weight of the Allium sativum extract composition, and/or
Between 1 and 9% by weight of the Primulae flos cum calycibus extract composition.

According to an embodiment of the invention, said cosmetic or dermatological composition comprises, by weight of composition:
Purified Water for pharmaceutical use: qs
A total of 20 mg of silver, up to 20% ionic silver and 80% colloidal silver, having a mean particle size of 0.8 nm;
0.9% Sambucus Nigra extract;
5% Hypericum perforatum extract;
1.5% Pelargonium sidoides dry extract;
3% Echinacea dry extract;
4% Propolis purified extract.

According to an embodiment of the invention, said cosmetic or dermatological composition comprises, by weight of composition:
Purified Water for pharmaceutical use: qs
A total of 20 mg of silver, up to 20% ionic silver and 80% colloidal silver, having a mean particle size of 0.8 nm;
0.9% Sambucus Nigra extract;
5% Primulae flos cum calycibus dry extract;
5% Hypericum perforatum dry extract;
1.5% Pelargonium sidoides dry extract;
3% Echinacea dry extract;
4% Propolis purified extract.

According to an embodiment of the invention, said cosmetic or dermatological composition comprises, by weight of composition:
A total of 20 mg of silver, up to 20% ionic silver and 80% colloidal silver, having a mean particle size of 0.8 nm.
0.9% Sambucus Nigra extract;
5% Hypericum perforatum dry extract;
1.5% Pelargonium sidoides dry extract;
3% Echinacea dry extract;
4% Propolis purified extract.

“Non therapeutic cosmetic or dermatological composition” means, within the meaning of this invention, an application that does not aim to treat a patient but a healthy individual.

Indeed, the cosmetic composition according to the invention does not act as a drug for the treatment of diseases, it improves the superficial visual appearance of inflamed skin.

Within the scope of the invention, the cosmetic composition reduces the visual signs of inflammation.

Preferably, said cosmetic or dermatological composition comprises at least one cosmetically or dermatologically acceptable excipient.

According to the invention, “cosmetically or dermatologically acceptable” means an excipient that is useful for the preparation of a cosmetic or dermatological composition, which is generally safe, non toxic and neither biologically or otherwise undesirable, and which is acceptable for cosmetic and dermatological use, including for topical application.

According to the invention, “extract” means a preparation derived from the listed plants. Preferably, said extracts are obtained by using a solvent.

“Dry extract” means an extract whose solvent has been evaporated and that is thus left with the plant's active ingredient or ingredients.

The cosmetic or dermatological compositions according to the invention can come in the forms that are traditionally known for topical administration, i.e. lotions, shampoos, balms, foams, gels, dispersions, emulsions, sprays, serums, masks, liquid solutions, creams or sticks with excipients that allow for in particular, for some of them, penetration to improve the active ingredients'properties and accessibility.

According to a third aspect, the invention relates to the non therapeutic cosmetic or dermatological use in healthy subjects of a composition comprising:
Silver in colloidal form;
Sambucus Nigra extract;
Hypericum perforatum extract;
Pelargonium sidoides extract.

Preferably, said composition for such cosmetic or dermatological use according to the invention comprises:
Between 20 mg and 250 mg of silver in colloidal form per liter of composition.

Preferably, said composition for such cosmetic or dermatological use according to the invention comprises:
Between 0.3 and 5% by weight of the Sambucus Nigra Extract composition.

Preferably, said composition for such cosmetic or dermatological use according to the invention comprises:
Between 0.5 and 11% by weight of Hypericum perforatum extract.

Preferably, said composition for such cosmetic or dermatological use according to the invention comprises:
Between 1 and 6% by weight of the Pelargonium sidoides Extract composition.

According to an embodiment, said composition for such cosmetic or dermatological use according to the invention comprises:
Between 20 mg and 250 mg of silver in colloidal form per liter of composition;

Between 0.3 and 5% by weight of the Sambucus Nigra Extract composition;
Between 0.5 and 11% by weight of the Hypericum perforatum extract composition;
Between 1 and 6% by weight of the Pelargonium sidoides Extract composition.

According to an embodiment, said composition for such cosmetic or dermatological use according to the invention further comprises:
Silver in ionic form;

According to an embodiment, said composition for such cosmetic or dermatological use according to this latter embodiment comprises:
Between 20 mg and 250 mg of total silver per liter of composition, distributed in
   i. Between 70 and 99.99% by total weight of silver, silver in colloidal form;
   ii. Between 0.01 and 30% by total weight of silver, silver in ionic form;

According to an embodiment, said composition for such cosmetic or dermatological use comprises:
Between 20 mg and 250 mg of total silver per liter of composition, distributed in
   i. Between 70 and 99.99% by total weight of silver, silver in colloidal form;
   ii. Between 0.01 and 30% by total weight of silver, silver in ionic form;
Between 0.3 and 5% by weight of the Sambucus Nigra Extract composition;
Between 0.5 and 11% by weight of the Hypericum perforatum extract composition;
Between 1 and 6% by weight of the Pelargonium sidoides Extract composition.

According to an embodiment, said composition for such cosmetic or dermatological use according to the invention further comprises:
Echinacea extract, and/or
Propolis and/or
Artemisia extract and/or
Allium sativum extract and/or
Primulae flos cum calycibus extract.

Preferably, said composition for such cosmetic or dermatological use according to the invention comprises:
Between 1 and 9% by weight of the Primulae flos cum calycibus Extract composition.

Preferably, said composition for such cosmetic or dermatological use according to the invention comprises:
Between 0.01 and 8% by weight of the Echinacea extract composition.

Preferably, said composition for such cosmetic or dermatological use according to the invention comprises:
Between 0.01 and 10% by weight of the Allium sativum extract composition.

Preferably, said composition for such cosmetic or dermatological use according to the invention comprises:
Between 0.01 and 7% by weight of the propolis composition.

Preferably, said composition for such cosmetic or dermatological use according to the invention comprises:
Between 0.5 and 3% by weight of the Artemisia extract composition.

Preferably, said composition for such cosmetic or dermatological use according to the invention comprises:
Between 0.01 and 8% by weight of the Echinacea extract composition, and/or
Between 0.01 and 7% by weight of the Propolis composition, and/or
Between 0.5 and 3% by weight of the Artemisia extract composition, and/or
Between 0.01 and 10% by weight of the Allium sativum extract composition, and/or
Between 1 and 9% by weight of the Primulae flos cum calycibus extract composition.

According to an embodiment of the invention, such cosmetic use reduces the visual signs of inflammation.

“Reduces the visual signs of inflammation” means that in healthy subjects with mild visual signs of inflammation, such as redness, the cosmetic composition according to the invention reduces such visibility. In such a case, the cosmetic composition according to the invention only acts on the visible signs of inflammation.

According to an embodiment of the invention, such cosmetic use prevents and/or dampens the intensity of skin redness and/or brightens and/or harmonizes the complexion and/or hides the redness on the skin surface, in healthy subjects.

According to an embodiment of the invention, said composition for such cosmetic or dermatological use comprises, by weight of composition:
Purified Water for pharmaceutical use: qs
A total of 20 mg of silver, up to 20% ionic silver and 80% colloidal silver, having a mean particle size of 0.8 nm;
0.9% Sambucus Nigra extract;
5% Hypericum perforatum extract;
1.5% Pelargonium sidoides dry extract;
3% Echinacea dry extract;
4% Propolis purified extract.

According to an embodiment of the invention, said composition for such cosmetic or dermatological use comprises, by weight of composition:
Purified Water for pharmaceutical use: qs
A total of 20 mg of silver, up to 20% ionic silver and 80% colloidal silver, having a mean particle size of 0.8 nm;
0.9% Sambucus Nigra extract;
5% Primulae flos cum calycibus dry extract;
5% Hypericum perforatum dry extract;
1.5% Pelargonium sidoides dry extract;
3% Echinacea dry extract;

4% Propolis purified extract.

According to an embodiment of the invention, said composition for such cosmetic or dermatological use comprises, by weight of composition:
A total of 20 mg of silver, up to 20% ionic silver and 80% colloidal silver, having a mean particle size of 0.8 nm.
0.9% Sambucus Nigra extract;
5% Hypericum perforatum dry extract;
1.5% Pelargonium sidoides dry extract;
3% Echinacea dry extract;
4% Propolis purified extract.

According to a fourth aspect, the invention relates to an inflammation treatment method, preferably induced by S. aureus comprising the administration to a person who needs it of an efficient quantity of the composition according to the invention.

According to a fifth aspect, the invention relates to a preparation process of a cosmetic or dermatological composition according to the invention, comprising at least a step of mixing the composition compounds.

In the description and in the following examples, unless otherwise specified, the percentages are percentages by weight and the value ranges written down in the form “between . . . and . . . ” include the specified minimum and maximum limit values. The examples below are presented for information only and are not restrictive of the field of the invention.

The following examples illustrate the invention without limiting its scope.

### FIGURES

FIG. 1 is a bar chart showing the IL-8 (pg/ml) concentrations following various treatments: 1—Composition according to example 1 (in liquid solution form); 2—Composition according to example 1 (in liquid solution form)+S. aureus; 3—Composition according to example 1 in cream form; 4—Composition according to example 1 in cream form+S. aureus.The data was derived from at least three independent experiments.

FIG. 2 is a bar chart showing the IP-10 (pg/ml) concentrations following: various treatments: 1-Composition according to example 1 (in liquid solution form); 2 Composition according to example 1 (in liquid solution form)+S. aureus;3-Composition according to example 1 in cream form; 4-Composition according to example 1 in cream form+S. aureus.The data was derived from at least three independent experiments.

FIG. 3 is a bar chart showing the IL-1β (pg/ml) concentrations following various treatments: 1—Composition according to example 1 (in liquid solution form); 2 Composition according to example 1 (in liquid solution form)+S. aureus; 3—Composition according to example 1 in cream form; 4—Composition according to example 1 in cream form+S. aureus. The data was derived from at least three independent experiments.

FIG. 4 is a bar chart showing the IL-6 (pg/ml) concentrations following various treatments: 1—Composition according to example 1 (in liquid solution form); 2—Composition according to example 1 (in liquid solution form)+S. aureus; 3—Composition according to example 1 in cream form; 4—Composition according to example 1 in cream form+S. aureus. The data was derived from at least three independent experiments.

### EXAMPLES

### Example 1: Composition Example

For 100 mL of composition, the composition comprises:
A total of 20 mg of silver, up to 20% ionic silver and 80% colloidal silver, having a mean particle size of 0.8 nm.
0.9% Sambucus Nigra extract.
5% Hypericum perforatum dry extract.
1.5% Pelargonium sidoides dry extract.
3% Echinacea dry extract.
4% Propolis purified extract.

### Example 2: Anti-Inflammatory Activity of the Composition in Example 1

### Materials and Methods

Human HT-29 epithelial cell line monolayers were grown in DMEM (Dulbecco's Modified Eagle's Medium, Gibco, UK), supplemented with 10% fetal bovine serum, at 37° C. and 5% of CO2. In about 90% of the monolayers, the cells were incubated with a 0.25% trypsin solution and 10 mM EDTA for 10 minutes at 37° C.

To determine the anti-inflammatory effect, eukariotic cells were grown in 24-well culture plates at a concentration of 2×10⁵ cells/ml. The medium was changed every 2 days over a total of 14 days, promoting not only the formation of a monolayer (3-4 days), but also cell receptor maturation. The monolayer obtained was washed twice with Phosphate buffered saline (PBS).

DMEM (1 ml) containing different preparations was added to each well and incubated for 20 hours at 37° C. The following preparations were used
Control DMEM;
DMEM with Staphylococcus aureus 1×10⁵ CFU/ml;
DMEM with 10% of the composition according to example 1 (in liquid solution form); DMEM with 10% of the composition according to example 1 (in liquid solution form) and Staphylococcus aureus 1×10⁵ CFU/ml;
DMEM with 10% of the composition according to example 1 in cream form;
DMEM with 10% of the composition according to example 1 in cream form and Staphylococcus aureus 1×10⁵ CFU/ml.

Following incubation, the acellular supernatant was used to determine the cytokine expression. IL-8, IP-10, IL-1β and IL-6 were tested by ELISA (enzyme-linked immunosorbent assays) according to manufacturing instructions (Diaclone, USA).

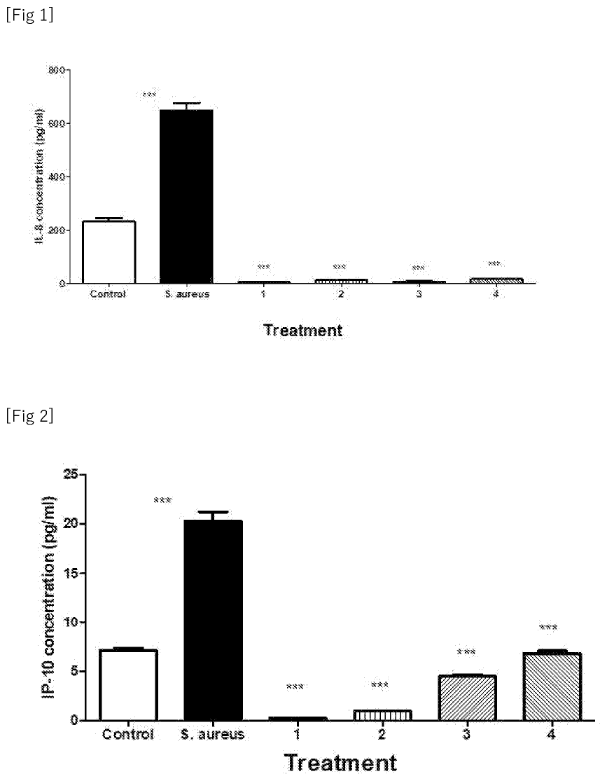

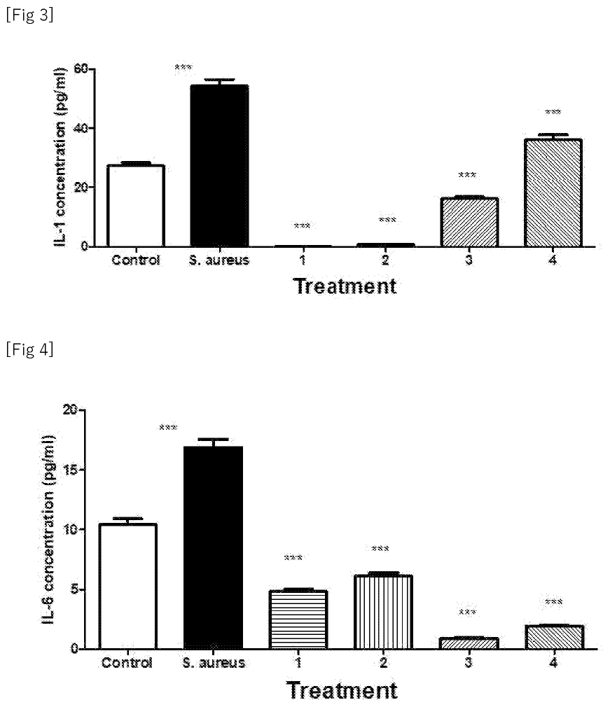

Findings The findings show that Staphylococcus aureus increases the concentration of pro-inflammatory cytokines compared to the cell line samples that were not treated in DMEM. A 3-fold increase was measured for IL-8 (FIG. 1) and IP-10 (FIG. 2). Levels about twice as high were reported for IL-18 (FIG. 3). The lowest increase, about 1.5-fold, was obtained for IL-6 (FIG. 4). This finding shows that in these experiments, IL-8 and IP-10 are the most sensitive markers of Staphylococcus aureus-induced inflammation.

The data revealed no over expression of IL-8 following treatment with the two compositions according to the invention, in cream and in liquid solution form (FIG. 1). No over expression was measured following the application of the products on cell lines with S. aureus-induced inflammation. The measured levels were similar.

The next tested marker was IP-10. The data revealed that better protection expressed by IP-10 over expression inhibition was reported following application of the composition according to example 1 when not in cream form (or in liquid solution form), compared to the cream (FIG. 2).

The same result was reported for the other tested marker-IL-1β-60 times lower (FIG. 3). The composition according to example 1 in cream form reduced IL-1β induction by about 1.5-fold.

For IL-6, the greatest reduction was obtained after application of the cream according to example 1 in cream form: about 9-fold (FIG. 4). The reduction of IL-6 following application of the composition according to example 1 was calculated to be 6-fold lower.

### Discussion

The findings show that the two preparations (the composition according to example 1 in liquid solution form and the composition according to example 1 in cream form) reduced the concentration of the four pro-inflammatory cytokines in both cases:
control samples (DMEM); and
cell lines in a state of inflammation induced by Staphylococcus aureus.

Based on the reported data, we can draw the conclusion that the ability of the compo sition according to example 1 in liquid solution to reduce the concentration of IL-8, IP-10, and IL-1β is greater compared to the effect of the composition according to example 1 in cream form. However, the effect of the composition according to example 1 in cream form proved to be greater on IL-6 compared to the composition according to example 1 that is not in cream form (in liquid solution form).

### REFERENCES

Agarwal, H., & Shanmugam, V. (2020). A review on anti-inflammatory activity of green synthesized zinc oxide nanoparticle: Mechanism-based approach. Bioorganic chemistry, 94, 103423.
Al-Daghri, N. M., Yakout, S., Al-Shehri, E., Al-Fawaz, H., Aljohani, N., & Al-Saleh, Y. (2014). Inflammatory and bone turnover markers in relation to PTH and vitamin D status among Saudi postmenopausal women with and without osteoporosis. International journal of clinical and experimental medicine, 7(9), 2812.
Dinarello, CA. Interleukin-1. Cytokine Growth Factor Rev 1997. 8:253-265.
Greten, F. R., & Grivennikov, S. I. (2019). Inflammation and cancer: triggers, mechanisms, and consequences. Immunity, 51(1), 27-41.
Gunalan, G., Vijayalakshmi, K., Saraswathy, A., Hopper, W., & Tamilvannan, T. (2014). Anti-inflammatory activities of phytochemicals from Bauhinia variegata Linn. leaf: An in silico approach. J. chem. pharm, 6(9), 334-48.
Kolb, M., Margetts, P. J., Anthony, D. C., Pitossi, F., & Gauldie, J. (2001). Transient expression of IL-1β induces acute lung injury and chronic repair leading to pulmonary fibrosis. The Journal of clinical investigation, 107(12), 1529-1536.
Liu, Q., Mazhar, M., & Miller, L. S. (2018). Immune and inflammatory responses to Staphylococcus aureus skin infections. Current dermatology reports, 7(4), 338-349.
Medzhitov, R. (2008). Origin and physiological roles of inflammation. Nature, 454(7203), 428-435.
Mueller, M., Hobiger, S., & Jungbauer, A. (2010). Anti-inflammatory activity of extracts from fruits, herbs and spices. Food chemistry, 122(4), 987-996.
Pathak, S., Goldofsky, E., Vivas, E. X., Bonagura, V. R., & Vambutas, A. (2011). IL-1β is overexpressed and aberrantly regulated in corticosteroid nonresponders with autoimmune inner ear disease. The Journal of Immunology, 186(3), 1870-1879.
Peiró, C., Lorenzo, Ó., Carraro, R., & Sánchez-Ferrer, C. F. (2017). IL-1β inhibition in cardiovascular complications associated to diabetes mellitus. Frontiers in pharmacology, 8, 363.
Sims, J. E., and D. E. Smith. 2010. The IL-1 family: regulators of immunity. Nat. Rev. Immunol. 10:89-102.
Singh, J. K., Simões, B. M., Howell, S. J., Farnie, G., & Clarke, R. B. (2013). Recent advances reveal IL-8 signaling as a potential key to targeting breast cancer stem cells. Breast Cancer Research, 15(4), 1-9.
Tatiya-Aphiradee, N., Chatuphonprasert, W., & Jarukamjorn, K. (2019). Anti-inflammatory effect of Garcinia mangostana Linn. pericarp extract in methicillin-resistant Staphylococcus aureus-induced superficial skin infection in mice. Biomedicine & Pharmacotherapy, 111, 705-713.

1-10. (canceled)

11. A composition for prophylaxis and/or treatment of inflammation comprising:
silver in colloidal form;
sambucus nigra extract;
hypericum perforatum extract; and
pelargonium sidoides extract.

12. A composition according to claim 11, wherein said silver in colloidal form has a particle size between 0.1 and 30 nanometers.

13. A composition according to claim 11, wherein the composition comprises:
between 20 mg and 250 mg of the silver in colloidal form per liter of the composition;
between 0.3 and 5% by weight of the sambucus nigra extract composition;
between 0.5 and 11% by weight of the hypericum perforatum extract composition; and
between 1 and 6% by weight of the pelargonium sidoides extract composition.

14. A composition according to claim 11, further comprising silver in ionic form.

15. A composition according to claim 14, wherein the composition comprises:
between 20 mg and 250 mg of total silver per liter of composition, distributed as:
between 70 and 99.99% of the silver in colloidal form by total weight of said silver; and
between 0.01 and 30% of the silver in ionic form by total weight of said silver;
between 0.3 and 5% by weight of the sambucus nigra extract composition;
between 0.5 and 11% by weight of the hypericum perforatum extract composition; and
between 1 and 6% by weight of the pelargonium sidoides extract composition.

16. A composition according to claim 11, further comprising:
echinacea extract; and/or
propolis, and/or
artemisia extract and/or
allium sativum extract and/or allium
primulae flos cum calycibus extract.

17. A composition according to claim 16, wherein the composition comprises:
between 0.01 and 8% by weight of the echinacea extract composition, and/or
between 0.01 and 7% by weight of the propolis composition, and/or
between 0.5 and 3% by weight of the artemisia extract composition, and/or
between 0.01 and 10% by weight of the allium sativum extract composition, and/or
between 1 and 9% by weight of the primulae flos cum calycibus extract composition.

18. A composition according to claim 11 wherein the composition is in the form of an ointment, cream, liquid solution, patch or gel.

19. A method for prophylaxis and/or treatment of inflammation comprising administering to a subject in need thereof a composition comprising:
silver in colloidal form;
sambucus nigra extract;
hypericum perforatum extract; and
pelargonium sidoides extract.

20. A method according to claim 19, wherein said silver in colloidal form has a particle size between 0.1 and 30 nanometers.

21. A method according to claim 19, wherein said composition comprises:
between 20 mg and 250 mg of the silver in colloidal form per liter of the composition;
between 0.3 and 5% by weight of the sambucus nigra extract composition;
between 0.5 and 11% by weight of the hypericum perforatum extract composition; and
between 1 and 6% by weight of the pelargonium sidoides extract composition.

22. A method according to claim 19, wherein the composition further comprises silver in ionic form.

23. A method according to claim 22, wherein the composition comprises:
between 20 mg and 250 mg of total silver per liter of composition, distributed as:
between 70 and 99.99% of the silver in colloidal form by total weight of said silver; and
between 0.01 and 30% of the silver in ionic form by total weight of said silver;
between 0.3 and 5% by weight of the sambucus nigra extract composition;
between 0.5 and 11% by weight of the hypericum perforatum extract composition; and
between 1 and 6% by weight of the pelargonium sidoides extract composition.

24. A method according to claim 19, wherein the composition further comprises:
echinacea extract; and/or
propolis, and/or
artemisia extract and/or
allium sativum extract and/or
primulae flos cum calycibus extract.

25. A method according to claim 24, wherein the composition comprises:
between 0.01 and 8% by weight of the echinacea extract composition, and/or
between 0.01 and 7% by weight of the propolis composition, and/or
between 0.5 and 3% by weight of the artemisia extract composition, and/or
between 0.01 and 10% by weight of the allium sativum extract composition, and/or
between 1 and 9% by weight of the primulae flos cum calycibus extract composition.

26. A method according to claim 19 wherein said inflammation is Staphylococcus aureus-induced inflammation.

27. A method according to claim 19 wherein the method comprises applying the composition to the skin in the form of an ointment, cream, liquid solution, patch or gel.

28. A cosmetic treatment method for reducing the visual signs of inflammation in a subject, comprising administering to the subject a composition comprising:
silver in colloidal form;
sambucus nigra extract;
hypericum perforatum extract; and
pelargonium sidoides extract.
